(19) United States
(12) Patent Application Publication
(10) Pub. No.: US 2025/0031645 A1
(43) Pub. Date: Jan. 30, 2025

(54) A METHOD FOR INCREASING THE DIGESTIBILITY AND/OR ASSIMILABILITY OF PLANTS

(71) Applicant: DANSTAR FERMENT AG, Zug (CH)

(72) Inventors: Olivier COR, Saint Jean de Boiseau (FR); Denis BEAUDET, Québec (CA); Julien SINDOU, Ramonville-Saint-Agne (FR)

(21) Appl. No.: 18/838,082

(22) PCT Filed: Feb. 14, 2023
(86) PCT No.: PCT/EP2023/053597

§ 371 Date: Aug. 13, 2024

(30) Foreign Application Priority Data
Feb. 14, 2022 (EP) 22305161.6

(51) Int. Cl. A01H 3/00

(52) U.S. Cl. CPC A01H 3/00

(57) ABSTRACT

The present disclosure concerns a method for increasing digestibility and/or assimilability and/or energy content and/or nutritional value of forage plants comprising contacting a plant, a plant part or a plant seed with at least one endophytic fungus species to colonize the roots of the plant or a plant grown from the plant seed. The present disclosure further relates to a method for increasing digestibility and/or assimilability and/or energy content and/or nutritional value of forage plants comprising contacting a plant, a plant part or a plant seed with at least one endophytic fungus species and a yeast to colonize the plant, the roots of the plant or a plant grown from the plant seed.

### TECHNOLOGICAL FIELD

The present disclosure relates to a method for increasing the digestibility and/or assimilability and/or energy content of plants, as for example forage plants used as animal feed.

### BACKGROUND

Arbuscular mycorrhizal fungi (AMF) are symbiotic microorganisms that colonize roots of most agricultural crops and are capable of forming a complex network of fungal hyphae that can be likened to an extension of plant root systems (Ryan & Graham 2002). Mycorrhizal infection may directly enhance plant mineral nutrition by increasing the volume of soil explored and by penetrating small soil cores with the thin diameter hyphae (Jakobsen et al. 2001). It has been well documented that the external hyphae of AMF can take up and deliver nutrients to the plants. There are also non-nutritional advantages for the host plants, as AMF can improve plants resistance to abiotic stressors, such as drought, salinity, heavy metals (Gianinazzi et al. 2010). AMF can promote plant growth and can be used to replace or complement mineral fertilizers.

Forages, which are composed of hays and silage are important feedstock for both milk-producing and meat-producing animals. In other words, forage is the basic feed for ruminants and their quality can have a significant impact on milk and meat production. Ruminants are mammals that are able to acquire nutrients from plant-based food containing cellulose by fermenting it in a specialized stomach (rumen) prior to digestion, principally through microbial actions. Ruminants include both domestic and wild species, such as bovines, goats, sheep, giraffes, deer, gazelles, and antelopes.

Nutritional quality of forage (which is composed of hays and silage) is determined by several parameters which are collectively known as digestibility. Digestibility is the relative amount of nutrients (nutritive substances), which are absorbed by the animal after feeding. Parameters that describe the digestibility of forages are, amongst other, Neutral Detergent Fiber (NDF), Acid Detergent Fiber (ADF) and Total Digestible Nutrients as percent of Dry Matter (TDN).

Many approaches have been taken to increase the digestibility, nutritional value or quality of forage and silage, but most rely on agronomic factors. Forage quality (i.e. its digestibility or nutritional value) is influenced by species selection, level of fertilization, maturity of the crop, plant part selected and, in corn, the relative development of the ear. Plant breeding of forages for example, has focused on lignin content which is a quality factor that is a negative indicator for forage digestibility. Genetic improvement to decrease lignin content was postulated to increase digestibility of forages, but it resulted in significant yield decreases (Pedersen, et al, 2005, Impact of reduced lignin on plant fitness Crop Sci. 45:812).

Accordingly, there is a need for improved methods to increase the digestibility of forage plants to render the plant material more easily assimilated by animals.

### BRIEF SUMMARY

The present disclosure relates to a method for increasing fiber digestibility, feed value, nutritional value, energy content and/or assimilability of forage plants used as animal feed.

The present disclosure relates to a method for increasing digestibility and/or assimilability and/or energy content of forage plants comprising contacting a plant, a plant part or a plant seed with at least one endophytic fungus species to colonize roots of the plant or a plant grown from the plant seed. The present disclosure further relates to a method for increasing silage digestibility and/or energy content comprising contacting a plant, a plant part or a plant seed with at least one endophytic fungus species to colonize the roots of the plant or a plant grown from the plant seed. The method of the present disclosure typically further comprises separately, simultaneously or sequentially contacting a plant, a plant part or a plant seed with a yeast. In an embodiment, the method of the present disclosure may further comprise simultaneously contacting a plant, a plant part or a plant seed with a yeast. In a further embodiment, the yeast is in the form of a live yeast, a dead or inactivated yeast, yeast cell walls, soluble yeast extract or yeast cell wall fractions. In a specific embodiment, the yeast is a dead or inactivated yeast. In an embodiment, the at least one endophytic fungus species is at least one arbuscular mycorrhizal fungus species. For example, the at least one arbuscular mycorrhizal fungus species is at least one strain of Rhizophagus species. In an embodiment, the at least one arbuscular mycorrhizal fungus species is at least one strain of Rhizophagus irregularis. In a further embodiment, the at least one strain of Rhizophagus irregularis is contacted with a plant seed. In an embodiment, the plant, plant part or plant seed is from alfalfa, clover, corn, rice, wheat, barley, oats, rye, millet, buckwheat, quinoa or sesame. For example, the plant seed is from alfalfa, clover or corn. In a further embodiment, the at least one strain of R. irregularis is present in an amount of about 0.001% to 50% by weight (w/w) of the combination, formulation or composition and the dead or inactivated yeast is present in an amount of about 0.1% to 99% by weight (w/w) of the combination, formulation or composition.

The present disclosure further provides a use of at least one strain of Rhizophagus and an inactivated yeast for increasing digestibility and/or assimilability and/or energy content of forage plants.

Also provided by the present disclosure is a use of at least one strain of Rhizophagus, an inactivated yeast and Bacillus velezensis for increasing digestibility and/or assimilability and/or energy content of forage plants.

The present invention thus provides a method for increasing digestibility and/or assimilability and/or energy content and/or nutritional value of forage plants comprising contacting a plant, a plant part or a plant seed with at least one endophytic fungus species to colonize roots of the plant or a plant grown from the plant seed, and separately, simultaneously or sequentially contacting the plant, the plant part or the plant seed with a yeast. The invention further provides a method for increasing silage digestibility and/or energy content and/or nutritional value comprising contacting a plant, a plant part or a plant seed with at least one endophytic fungus species to colonize the roots of the plant or a plant grown from the plant seed, and separately, simultaneously or sequentially contacting the plant, the plant part or the plant seed with a yeast. The invention also provides use of at least one endophytic fungus species and an inactivated yeast for increasing digestibility and/or assimilability and/or energy content and/or nutritional value of forage plants. The invention additionally provides use of at least one endophytic fungus species an inactivated yeast and Bacillus velezensis for increasing digestibility and/or assimilability and/or energy content and/or nutritional value of forage plants.

The invention further provides a forage plant seed inoculated with a combination, formulation or composition comprising at least one endophytic fungus species and yeast. The invention additionally provides a method for inoculating a forage plant seed, comprising contacting said forage plant seed with a combination, formulation or composition comprising at least one endophytic fungus species and yeast. The invention also provides a method of growing a forage plant, comprising sowing an inoculated forage plant seed of the invention and growing a forage plant from said seed. The invention further provides a method of producing silage, wherein the silage is produced from a forage plant grown from an inoculated forage plant seed of the invention.

### General Definitions

In the following description and examples, a number of terms are used. In order to provide a clear and consistent understanding of the specification and claims, including the scope to be given to such terms, the following definitions are provided. Unless otherwise defined herein, all technical and scientific terms used have the same meaning as commonly understood by one of ordinary skill in the art to which this invention belongs. The disclosures of all publications, patent applications, patents and other references are incorporated herein in their entirety by reference.

The terms “comprising” or “to comprise” and their conjugations, as used herein, refer to a situation wherein said terms are used in their non-limiting sense to mean that items following the word are included, but items not specifically mentioned are not excluded. It also encompasses the more limiting verb “to consist essentially of” and “to consist of”.

Reference to an element by the indefinite article “a” or “an” does not exclude the possibility that more than one of the elements is present, unless the context clearly requires that there be one and only one of the elements. The indefinite article “a” or “an” thus usually means “at least one”.

As used in the present disclosure, the term “digestibility” is a criterion which defines the degree to which organic matter can be digested by an animal. Digestibility is the relative amount of nutrients (nutritive substances), which are absorbed by the animal after feeding. In the plant kingdom (fodder for example) this criterion generally decreases, as the level of lignin in a plant increases. The vegetative parts of plants have much higher digestibility than the stalks for example.

As used herein, the term “digestibility and/or assimilability of forage plants” means the degree to which the forage plants (e.g. plant parts, fibers or grains) can, after the treatment of the present disclosure, present higher content of soluble carbohydrates, fatty acids and amino acids that can be assimilated by the animal. An increase in the digestibility and assimilability of plants, plant parts, fibers and/or grains results in an increase in the proportion of available nutrients extracted from the forage plants that will be consumed. More generally, “an increase in digestibility and assimilability of plant fibers” results in an increase in the proportion of available nutrients from the forage plants which will be consumed by animals. It also refers to increased nutritional quality or nutritional value of the forage plants.

As used herein, the term “increasing” quality, nutrient availability, energy content, digestibility or assimilability of forage plants means that the forage plants (including the plant fibers) have more of the quality, nutritional value, energy content, digestibility or assimilability than the forage plants would have had it if it had not been treated by methods of the present disclosure. “Nutrient availability” as used herein refers to the amount of soluble nutrients made available after the treatment of the present disclosure.

As used herein, the term “increased energy content” refers to increased nutritional quality and/or nutritional value of forage plants leading, for example, to an increase in net energy of lactation. As previously explained, this nutrient availability, nutritional quality and/or nutritional value is determined by any method known in the art to quantify the nutritional quality and/or nutritional value.

The term “forage plants” as used herein, means any plant cultivated for its fibers and grains which can be used in animal feed. Non-limitative examples of forage plants are grass, hay, alfalfa, straw, clover, grains, maize, rice, wheat, barley, oats, pea, sorgum, lentil, rye, millet, buckwheat, quinoa or sesame, brassicas but also other vegetables, or mixture thereof.

In the context of the present disclosure, the term “fibers” or “plant fibers” of forage plants is meant a natural plant substance constituted mainly by a carbohydrate polymer. Non-limitative examples of plant fibers are the celluloses, hemicelluloses, pectins, proteoglycans, lignin, etc.

As used herein, the terms “plant” and “plant part” means all plants and plant populations such as desired and undesired wild plants or crop plants (including naturally occurring crop plants). Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes.

As used herein, the term “forage” typically refers to any plant material eaten by grazing livestock, particularly ruminants.

The term “silage” as used herein typically refers to any grass or other green fodder stored for use as animal feed and typically includes all types of fermented agricultural products.

The term “contacting” a plant, a plant part or a plant seed as described in the present disclosure includes any method by which an at least one endophytic fungus species (which can be combined with a yeast and/or a bacteria) is brought into contact with the plant, plant part or plant seed thereof. Some non-limiting examples of contacting a plant a part or a plant seed thereof include spraying, dusting, sprinkling, scattering, misting, atomizing, broadcasting, soaking, soil injection, soil incorporation, drenching (e.g., soil treatment), pouring, coating, leaf or stem infiltration, side dressing or seed treatment, and the like, and combinations thereof. These and other procedures for contacting a plant a part or a plant seed thereof with compound(s), composition(s) or formulation(s) are well-known to those skilled in the art.

As used herein, the term “simultaneously” means that an at least one endophytic fungus species of the present disclosure and a yeast can be delivered to a plant, a plant part or a plant seed at the same time or substantially at the same time via the same mode of application.

As used herein, the term “separately” means that an at least one endophytic fungus species of the present disclosure and a yeast can be delivered to a plant, a plant part or a plant seed at the same time or substantially at the same time via a different mode of application.

As used herein, the term “sequentially” means that an at least one endophytic fungus species of the present disclosure and a yeast are delivered to a plant, a plant part or a plant seed at different times (i.e. an at least one endophytic fungus species of the present disclosure can be delivered before or after the yeast), the mode of application being identical or different. As used herein, the term “yeast” denotes a yeast strain, i.e. a yeast obtained by culturing the yeast strain. The yeast can be in live or dead (i.e. inactive or inactivated yeast) form, and in the form of yeast derivatives such as a soluble yeast extract, yeast cell walls and purified fractions of yeast cell walls, or mixtures thereof.

### DETAILED DESCRIPTION

The present disclosure provides a method for increasing the digestibility and/or assimilability and/or energy content (i.e. nutritional value) of forage plants. It has been demonstrated that certain fungus species possesses the property of increasing the digestibility and/or nutritional value of such forage plants, including all types of plant fibers of forage plants.

In an embodiment, the present disclosure relates to a method for increasing digestibility and/or assimilability and/or energy content of forage plants comprising contacting a plant, a plant part or a plant seed with an at least one endophytic fungus species to colonize the plant, roots of the plant or a plant grown from the plant seed.

In another embodiment, the present invention relates to a method for increasing digestibility and/or assimilability and/or energy content and/or nutritional value of forage plants comprising contacting a plant, a plant part or a plant seed with at least one endophytic fungus species to colonize the plant, the roots of the plant or a plant grown from the plant seed which method further comprises contacting the plant, plant part or plant seed separately, simultaneously or sequentially with a yeast. In an embodiment, the at least one endophytic fungus species and the yeast are used simultaneously to contact the plant, plant part or plant seed.

In an embodiment, the present disclosure relates to a method for increasing digestibility and/or assimilability and/or energy content of forage plants comprising contacting a plant, a plant part or a plant seed with a combination of at least one endophytic fungus species and a yeast to colonize the plant, roots of the plant or a plant grown from the plant seed and a yeast. In an embodiment, the at least one endophytic fungus species and the yeast are applied to a plant seed. In an embodiment, the present invention is directed to a method for increasing silage digestibility of a forage crop comprising contacting a plant, a plant part or a plant seed with a combination of an at least one endophytic fungus species and a yeast to colonize the plant, roots of the plant or a plant grown from the plant seed and a yeast.

In an embodiment, the present invention is directed to use of at least one endophytic fungus species and an inactivated yeast for increasing digestibility and/or assimilability and/or energy content and/or nutritional value of forage plants. In a further embodiment, the invention is directed to use of at least one endophytic fungus species, an inactivated yeast and Bacillus velezensis for increasing digestibility and/or assimilability and/or energy content and/or nutritional value of forage plants. In an embodiment, the present invention is directed to a forage plant seed inoculated with a combination, formulation or composition comprising at least one endophytic fungus species and yeast. In a further embodiment, the present invention is directed to a method for inoculating a forage plant seed, comprising contacting said forage plant seed with a combination, formulation or composition comprising at least one endophytic fungus species and yeast.

In a further embodiment, the present invention is directed to a method of growing a forage plant, comprising sowing an inoculated forage plant seed according to the invention and growing a forage plant from said seed. In a further embodiment, the present invention is directed to a method of producing silage, wherein the silage is produced from a forage plant grown from an inoculated forage plant seed of the invention.

In a further embodiment, the present disclosure is directed to silages for feeding animals, produced from a plant, a plant part or a plant seed treated with a combination of an at least one endophytic fungus species and a yeast prior to producing silages, where the treated silage displays increased digestibility and energy content.

By an increase in digestibility and/or assimilability and/or energy content and/or nutritional value of forage plants, it is meant that contacting a plant, a plant part or a plant seed with the at least one endophytic fungus species or combination thereof with a yeast results in higher digestibility, assimilability, energy content and/or nutritional value of the forage plants as compared to forage plants not contacted with the endophytic fungus species or combination thereof with a yeast. Similarly, by an increase in silage digestibility and/or energy content and/or nutritional value, it is meant that contacting a plant, a plant part or a plant seed with the at least one endophytic fungus species or combination thereof with a yeast results in a silage being obtained from plants that has a higher digestibility, energy content and/or nutritional value as compared to a silage obtained from plants where a plant, a plant part or a plant seed is not contacted with the endophytic fungus species or combination thereof with a yeast.

Examples of endophytic fungus species that can be used in the context of the present disclosure are fungus of the orders Sebecinales (as, for example, Serendipita species) or arbuscular mycorrhizal fungus. An arbuscular mycorrhizal fungus is a type of mycorrhizae in which the fungus penetrates the cortical cells of the roots of a vascular plant. Arbuscular mycorrhizal fungi help plants capture nutrients such as phosphorus, sulfur, nitrogen and soil micronutrients. The term “arbuscular mycorrhizal fungus” as used herein is intended to refer to fungus from the Glomeromycota phylum. In an embodiment, the arbuscular mycorrhizal fungus is a Rhizophagus species. Preferably, the at least one endophytic fungus is a strain of Rhizophagus irregularis. More particularly, a preferred strain in the context of the present disclosure is R. irregularis strain 57891 which has been deposited on the 12th Jan. 2023 according to the Budapest Treaty under accession number 57891 with the Belgium Coordinated Collection of Micro-organisms (BCCM), Université catholique de Louvain, Mycothèque de l′Université catholique de Louvain (MUCL), Croix de Sud 2, box L7.05.06, 1348 Louvain-la-neuve, Belgium. The strain 57891 is the active principle of the LALRISE® PRIME product developed by the company Lallemand Inc.

According to an embodiment of the present disclosure, the yeast is chosen from yeasts of the genus Saccharomyces, and in particular the yeast is a strain of Saccharomyces cerevisiae. Other suitable yeast strains include non-Saccharomyces genus, as for example, but not limited to Kluyveromyces, Hanseniaspora, Metschnikowia, Pichia, Starmerella, Torulaspora, Candida, Brettanomyces, Schizosaccharomyces or Lachancea.

In the context of the present invention, the yeast can be a live yeast in the form of a dry yeast. A dry yeast, produced by freeze-drying, fluidized-bed drying, drum drying or spray-drying is characterized by a low water content.

According to another embodiment, the yeast strain is used in the form of dead yeast. A dead yeast may also be called “deactivated yeast” or “inactive yeast” or “inactivated yeast”. It is a yeast whose metabolism is irremediably stopped. A dead yeast may be obtained by techniques well known to those skilled in the art, such as a heat treatment of the yeast, a treatment consisting in subjecting the yeast to several successive freezing and thawing cycles, a treatment by irradiation, a treatment by atomization or a combination of these treatments. An inactive yeast is generally in dry form.

In another embodiment, the yeast used in the context of the present disclosure is in the form of a yeast cell derivative. This derivative is chosen from a soluble yeast extract, yeast cell walls and yeast cell wall fractions that can be purified or not.

In a preferred embodiment, the yeast used in the context of the present disclosure is in the form of a dead yeast (i.e. inactive or inactivated yeast) and is from S. cerevisiae.

In a preferred embodiment, the at least one endophytic fungus species can be used simultaneously or in combination with a dead (or inactivated) yeast. When at least one strain of R. irregularis is used in combination with a yeast (as for example a dead yeast), the at least one strain of R. irregularis can be present in an amount of about 0.00001% to 99.9% by weight (w/w) of the combination or composition. When present in the combination, formulation or composition, the yeast (as for example a dead yeast) can be present in an amount of about 0.1% to 99.9999% by weight (w/w) of the combination, formulation or composition. In certain embodiments, the at least one strain of R. irregularis is present in an amount of about 0.00001% to 95%, about 0.00001% to 90%, about 0.00001% to 85%, about 0.00001% to 80%, about 0.00001% to 75%, about 0.00001% to 70%, about 0.00001% to 65%, about 0.00001% to 60%, about 0.00001% to 55%, about 0.00001% to 50%, about 0.00001% to 45%, about 0.00001% to 40%, about 0.00001% to 35%, about 0.00001% to 30%, about 0.00001% to 25%, about 0.00001% to 20%, about 0.0001% to 20%, 0.001% to 20%, 0.001% to 30%, 0.001% to 40%, 0.001% to 50%, about 0.01% to 50%, about 0.01% to 40%, about 0.01% to 30%, about 0.01% to 20%, about 0.01% to 10%, about 0.01% to 5%, about 0.1% to 10%, about 0.1% to 15%, about 0.1% to 20%, about 0.1% to 25%, about 0.1% to 30%, about 0.1% to 35%, about 0.1% to 40%, about 0.1% to 45% or about 0.1% to 50% by weight (w/w) of the combination, formulation or composition. In some embodiments, the yeast (as for example a dead yeast) is present in an amount of about 0.1% to 95%, about 0.1% to 90%, about 0.1% to 85%, about 0.1% to 80%, about 0.1% to 75%, about 0.1% to 70%, about 0.1% to 65%, about 0.1% to 60%, about 0.1% to 55%, about 0.1% to 50%, about 0.1% to 45%, about 0.1% to 40%, about 0.1% to 35%, about 0.1% to 30%, about 0.1% to 25%, about 0.1% to 20%, about 0.1% to 10% or about 0.1% to 5% by weight (w/w) of the combination, formulation or composition.

According to an embodiment of the present disclosure, the arbuscular mycorrhizal fungus/inactivated yeast weight ratio is between 0.001 and 100, preferably between 0.01 and 75, 0.02 and 50, 0.05 and 25, 0.05 and 20, 0.05 and 15, 0.05 and 10 and more preferably the weight ratio is 0.01; 0.02; 0.03; 0.04; 0.05; 0.06; 0.07; 0.08; 0.09; 0.1, 0.5; 1; 2; 3; 4; 5; 6; 7; 8; 9; 10; 20; 30; 40; 50; 60; 70; 80; 90 or 100. In a preferred embodiment, the weight ratio is between 0.05 and 10.

Although the combination, formulation or composition of the present disclosure can be applied to any portion of a plant or a plant part such as foliage, to the soil (spraying, spreading, sprinkling, in the seed drill or in the open field), by root dipping, by seed treatment or by incorporation into a cultivation support or by any means which makes it possible to bring in contact arbuscular mycorrhizal fungus and the inactivated yeast with the roots of a plant, immediately or in the close future, it is especially effective as a seed treatment, dressing and/or coating. For example, when the at least one strain of R. irregularis is employed as a seed treatment, dressing or coating, it is generally applied to the seed at a rate of from about 0,0001 to about 10 000 spores or propagules/g of seeds, and preferably at a rate of from about 0.001 to about 9000 spores or propagules/g of seeds, about 0.01 to about 8000 spores or propagules/g of seeds, about 1 to about 7000 spores or propagules/g of seeds, about 10 to about 6000 spores or propagules/g of seeds, about 50 to about 5000 spores or propagules/g of seeds, about 100 to about 5000 spores or propagules/g of seeds, about 200 to about 5000 spores or propagules/g of seeds, about 300 to about 5000 spores or propagules/g of seeds, about 400 to about 5000 spores or propagules/g of seeds, about 500 to about 5000 spores or propagules/g of seeds, about 600 to about 5000 spores or propagules/g of seeds, about 700 to about 5000 spores or propagules/g of seeds, about 800 to about 5000 spores or propagules/g of seeds, about 900 to about 5000 spores or propagules/g of seeds, about 1000 to about 5000 spores or propagules/g of seeds, about 1500 to about 5000 spores or propagules/g of seeds or about 2000 to about 5000 spores or propagules/g of seeds. The term “propagules” as used herein is typically used to denote simultaneously the spores, the vesicles and the fragments of roots containing vesicles, since all these structures serve to propagate the species.

According to an embodiment, the composition of the present invention may be in a dry mixture, in a wettable powder, granule or liquid form. The combination of the at least one endophytic fungus species and a dead yeast may further comprise an agriculturally acceptable carrier. The term “agriculturally acceptable carrier” as used herein means an inert, solid or liquid, natural or synthetic, organic or inorganic substance, which is mixed or combined with the at least one endophytic fungus species and a dead yeast, for better applicability on plants, plant parts or plant seeds thereof. It covers all carriers that are ordinarily used in (bio) fungicide formulation technology including, but not limited to, water, protective colloids, binders, salts, buffers, diluents, minerals, fillers, colorants, defoamers, adhesives, fixatives, tackifiers, resins, preservatives, stabilizers, fertilizers, anti-oxidation agents, gene activators, thickeners, plasticizers, siccatives, surfactants, dispersants, alcohols, complex formers, wetting agents, waxes, solvents, emulsifiers, mineral or vegetable oils, sequestering agents, and derivatives and/or mixtures thereof.

According to a particular embodiment of the present disclosure, the method for increasing digestibility and/or assimilability and/or energy content of forage plants comprising contacting a plant, a plant part or a plant seed with a combination of at least one endophytic fungus species and a yeast may further comprise the addition of other microorganisms such as at least one bacterium species such as, for example, plant-growth promoting rhizobacteria (PGPR). For example, the PGPR may be selected from genera including, but not limited to, Actinobacter, Alcaligenes, Bacillus, Burkholderia, Buttiauxella, Enterobacter, Klebsiella, Pseudomonas, Rahnella, Ralstonia, Rhizobium, Serratia, Stenotrophomonas, Paenibacillus and Lysinibacillus. In a preferred embodiment, Bacillus velezensis can be used.

According to a particular embodiment of the present disclosure, the method for increasing digestibility and/or assimilability and/or energy content of forage plants comprising contacting a plant, a plant part or a plant seed with a combination of at least one endophytic fungus species and a yeast may further comprise the addition of other microorganisms such as lactic acid bacteria or yeast during the silage period.

In the context of the present disclosure, the increase in digestibility and/or assimilability and/or energy content and/or nutritional value of forage plants can be seen, for example, by an increase in water-soluble carbohydrates (WSC-sugar), an increase in ethanol soluble carbohydrates (ECS-sugar), an increase in crude proteins (% CP), an increase in digestible fiber (IVTDMD), an increase in Relative Feed Value (RFV), a decrease in undigestible fiber (uNDF), an increase in Neutral Detergent Fiber Digestibility (NDFd), a decrease in Neutral Detergent Fibers (NDF), a decrease in Acid Detergent Fibers (ADF) or a decrease in lignin content.

In the context of the present disclosure, the increase in silage digestibility and/or energy content and/or nutritional value can be seen by an increase in water-soluble carbohydrates (WSC-Sugar), an increase in ethanol soluble carbohydrates (ESC-Sugar), an increase in the concentration of soluble fiber, an increase in calcium content, an increase in fatty acids total, an increase in crude proteins, an increase in the amount of net energy of lactation or an increase in the milk productivity, and increase in crude proteins (% CP), an increase in Relative Feed Value (RFV) or a decrease in Acid Detergent Fibers (ADF).

The following example serves to further describe and define the invention and is not intended to limit the invention in any way.

### Examples

### Example 1: Effect of Rhizophagus irregularis in Combination with Inactive Yeast on Alfalfa and Red Clover Forage Crop

Studies were carried out to assess the effects of seed inoculation by an arbuscular mycorrhizal fungus and inactived (inactivated) yeast on growth parameters, fiber digestibility and the energy content of alfalfa and red clover forage cropping.

The forage crops investigated were alfalfa (Medicago sativa, Isabelle variety) and red clover (Trifolium prate, Bearcat variety). Alfalfa and red clover field trials were conducted at different locations. Seeds of the two forage crops were inoculated with the mycorrhizal fungi R. irregularis strain 57891 (BCCM) (LALRISE® PRIME at a concentration of 2000 spores/g in an amount of at least 0.5% by weight (w/w) of the composition or formulation) in combination with inactivated or dead yeast (Lallemand, in an amount of at least 10% by weight (w/w) of the composition or formulation) at a label rate of 100 g/ha (1.5 oz per acre or 6.67 g of inoculum/kg of seeds). The seeds were mixed with the dry microbial product. Non-mycorrhizal alfalfa and red clover seeds without inactivated yeast were used as negative control.

Inoculated and non-inoculated alfalfa and red clover seeds were sown in 4 blocks of 7.5 square meter (per treatment) with a planting rate of 15 kg/ha. Blocks were arranged in randomized complete field trials.

Trials were harvested five times (i.e. up to five cuts depending on the tested parameters, forage crop and location) using commercial equipment at standard timings for alfalfa and red clover forage crops. Both inoculated and non-inoculated forage plants were harvested separately. Representative samples were taken for subsequent analysis. The following quality parameters were evaluated by standard methods at the first, second, third or fourth cuttings (depending of the tested parameters, forage crop or location) after treatment: yield, plant height, dry matter, lignin content (in %), fiber digestibility measurements (Neutral Detergent Fiber digestibility (NDFd), In Vitro Total Dry Matter Digestibility (IVTDMD at 30 hours and 48 hours), Undigestible Fiber (uNDF at 120 hours), Net Energy of Lactation (NEL), soluble carbohydrates and crude proteins).

The effect of R. irregularis and inactivated yeast on dry matter and yield of alfalfa are summarized in Table 1 below. Adding R. irregularis and inactivated yeast significantly increased dry matter by 7.4% (17.5 vs 16.3%, p=0.064) and yield by 3.6% (3371 vs 3255 kg/ha, p=0.08).

TABLE 1
Effect of R. irregularis and inactivated yeast
on dry matter (DM) and yield of alfalfa plants

| | Medicago sativa | Medicago sativa |
|---|---|---|
| | Alfalfa | Alfalfa |
| | Isabella | Isabella |
| | Cut 2 and 3 | Cut 2 |
| | Yield 100% DM | DM |
| | kg/ha | % |
| 1 Negative control | 3255 b | 16.3 b |
| 2 R. irregularis + inactive yeast | 3371 a | 17.5 a |
| Tukey's HSD P = .10 | 105.4 | 1.00 |
| Standard Deviation | 63.3 | 0.60 |
| CV | 1.91 | 3.58 |
| Replicate F | 22.018 | 2.472 |
| Replicate Prob(F) | 0.0152 | 0.2384 |
| Treatment F | 6.680 | 8.232 |
| Treatment Prob(F) | 0.0815 | 0.0641 |

The effect of R. irregularis and inactivated yeast on concentrations of soluble carbohydrates, crude proteins, undigestible fiber (uNDF at 120 hours) and lignin content of alfalfa plants are summarized in Table 2 below. The inoculation of alfalfa seeds with R. irregularis and inactivated yeast significantly increased, at the first cutting, the water-soluble carbohydrates (WSC-Sugar) by 7.6% (9.16 vs 8.51%, p=0.09) and the ethanol soluble carbohydrates (ESC-Sugar) by 15.1% (8.29 vs 7.20%, p=0.01) of alfalfa fibers. At the second cutting, the concentration of crude proteins (CP) significantly increased by 5.1% (26.6 vs 25.3%, p=0.07). At the third cutting, a significant increase in water soluble carbohydrates (WSC-Sugar) of 22% (5.16 vs 4.23%, p=0.08) and ethanol soluble carbohydrates (ESC-Sugar) of 36.5% (3.48 vs 2.55%, p=0.08) were observed. The amount undigestible fiber (uNDF at 120 hours) was quantified at the third cutting. Results showed a significant decrease in undigestible fiber by 10.6% (18.5 vs 20.7%, p=0.057). The content of lignin was analyzed at the fourth cutting. The inoculation of the alfalfa seeds with R. irregularis and inactivated yeast significantly decreased the lignin content by 6.8% (4.7 vs 4.4%, p=0.06).

TABLE 2
Effect of R. irregularis and inactivated yeast on concentrations of soluble carbohydrates, crude proteins,
undigestible fiber (uNDF at 120 hours) and lignin content of alfalfa plants according to the different cuts

| | Medicago | Medicago | Medicago | Medicago | Medicago | Medicago | Medicago |
|---|---|---|---|---|---|---|---|
| | sativa | sativa | sativa | sativa | sativa | sativa | sativa |
| | Cut 1 | Cut 1 | Cut 2 | Cut 3 | Cut 3 | Cut 3 | Cut 3 |
| | WSC-Sugar | ESC-Sugar | CP | WSC-Sugar | ESC-Sugar | uNDF-120 h | Lignin |
| | %; 0; 100 | %; 0; 100 | %; 0; 100 | %; 0; 100 | %; 0; 100 | %; 0; 100 | %; 0; 100 |
| 1 Negative | 8.51b | 7.20b | 25.3b | 4.23b | 2.55b | 20.7a | 4.7a |
| control | | | | | | | |
| 2. R. | 9.16a | 8.29a | 26.6a | 5.16a | 3.48a | 18.5b | 4.4b |
| irregularis | | | | | | | |
| and | | | | | | | |
| inactivated | | | | | | | |
| yeast | | | | | | | |
| Tuke''s HSD | 0.623 | 0.441 | 1.12 | 0.854 | 0.854 | 1.73 | 0.26 |
| P = .10 | | | | | | | |
| Standard | 0.374 | 0.265 | 0.67 | 0.513 | 0.513 | 1.04 | 0.16 |
| Deviation | | | | | | | |
| CV | 4.24 | 3.42 | 2.59 | 10.93 | 17.03 | 5.32 | 3.47 |
| Replicate F | 2.394 | 29.883 | 4.719 | 5.135 | 5.135 | 7.970 | 1.678 |
| Replicate | 0.2461 | 0.0098 | 0.1174 | 0.1061 | 0.1061 | 0.0610 | 0.3406 |
| Prob(F) | | | | | | | |
| Treatment F | 6.080 | 33.849 | 7.511 | 6.505 | 0.605 | 9.129 | 8.593 |
| Treatment | 0.0904 | 0.0101 | 0.0713 | 0.0839 | 0.0839 | 0.0567 | 0.0609 |
| Prob(F) | | | | | | | |

After the winter, both inoculated and non-inoculated alfalfa plants were harvested separately. The concentration of crude proteins (CP), the Neutral Detergent Fibers (NDF) and the Relative Feed Value (RFV) were evaluated according to standard methods known in the art.

The results analyzed after the second cutting in July are summarized in the Table 2 (a) below. In brief, the inoculation of alfalfa seeds with R. irregularis and inactivated yeast carried out in the previous year significantly increased, after the winter period, the concentration of crude protein content by 3.3% (24.8 vs 24.0%) and the Relative Feed Value by 3.5% (149 vs 144%). Further, the results show a significant decrease in Neutral Detergent Fibers (NDF) by 2.2% (41.4 vs 42.3%).

TABLE 2(a)
Effect of R. irregularis and inactivated yeast on concentrations
of crude proteins, Neutral Detergent Fibers (NDF) and the Relative
Feed Value of alfalfa plants one year after the inoculation

| | Medicago | Medicago | Medicago |
|---|---|---|---|
| | sativa | sativa | sativa |
| | Cut 2 | Cut 2 | Cut 2 |
| | CP | NDF | RFV |
| | %; 0; 100 | %; 0; 100 | %; 0; 100 |
| 1 Negative | 24.0 b | 42.3 a | 144 b |
| control | | | |
| 2. R. irregularis | 24.8 a | 41.4 b | 149 a |
| and inactivated | | | |
| yeast | | | |
| Tukey's | 0.31 | 0.78 | 0.7 |
| HSD P = .10 | | | |
| Standard | 0.19 | 0.47 | 0.4 |
| Deviation | | | |
| CV | 0.76 | 1.12 | 0.28 |
| Replicate F | 7.361 | 20.857 | 549.000 |
| Replicate | 0.0676 | 0.0164 | 0.0001 |
| Prob(F) | | | |
| Treatment F | 39.361 | 6.921 | 243.000 |
| Treatment | 0.0082 | 0.0783 | 0.0006 |
| Prob(F) | | | |

The various effect of the inoculation of R. irregularis and inactivated yeast on red clover seeds are summarized in Table 3 below. Adding the mycorrhizal fungi and inactivated yeast significantly increased the amount of net energy of lactation (NEL) by 5.6% (1.33 vs 1.26 mcal/kg, p=0.089). Further, the concentration of the lignin content as dry matter percent and as neutral detergent fiber percent significantly decreased by 15.7% (7.5 vs 8.9%, p=0.095) and 10.7% (15.0 vs 16.8%, p=0.002), respectively. The fiber digestibility was also estimated at third cutting and the results indicated an increase in digestible fiber of 4.0% (59.1 vs 56.8%, p=0.065) at 30 hours. Finally, a higher leaf/stem ratio was observed (24 vs 26.5 cm in average height, p=0.015; +10%).

TABLE 3
Effect of R. irregularis and inactivated yeast on the amount of net energy of
lactation (NEL), lignin content (as dry matter and as neutral detergent fiber) and
neutral detergent fiber (NDFd) of red clover plants according to different cuts

| | Trifolium prate | Trifolium prate | Trifolium prate | Trifolium prate | Trifolium prate |
|---|---|---|---|---|---|
| | Cut 2 | Cut 2 | Cut 2 | Cut 3 | Cut 3 |
| | NEL | Lignin | Lignin NDF | Leaf/stem ratio | NDFd-30 h |
| | mcal/kg | % | % | cm | % |
| 1 Negative | 1.26b | 8.9a | 16.8a | 26.5a | 56.83b |
| control | | | | | |
| 2. R. irregularis | 1.33a | 7.5b | 15.0b | 24.0b | 59.07a |
| and inactivated | | | | | |
| yeast | | | | | |
| Tuke''s HSD | 0.069 | 1.37 | 0.42 | 1.18 | 1.843 |
| P = .10 | | | | | |
| Standard | 0.041 | 0.82 | 0.25 | 0.71 | 1.107 |
| Deviation | | | | | |
| CV | 3.19 | 10.01 | 1.58 | 2.8 | 1.91 |
| Replicate F | 3.336 | 5.812 | 179.194 | 6.333 | 0.450 |
| Replicate | 0.1744 | 0.0912 | 0.0007 | 0.0819 | 0.7354 |
| Prob(F) | | | | | |
| Treatment F | 6.139 | 5.822 | 94.513 | 25.000 | 8.127 |
| Treatment | 0.0895 | 0.0948 | 0.0023 | 0.0154 | 0.0651 |
| Prob(F) | | | | | |

As done for alfalfa, clover plants were also analyzed after the winter period to determine if the effect of the treatment on the plants was still present. Both inoculated and non-inoculated clover plants were harvested separately and the Neutral Detergent Fiber Digestibility (NDFd_30 h and NDFd_48 h) was determined using standard method in the art.

The results were analyzed after the third cutting in August and it was observed that adding the mycorrhizal fungi and inactivated yeast significantly increased the Neutral Detergent Fiber Digestibility (NDFd) at 30 and 48 hours by 2.6% (59.73 vs 58.24%) and 3.1% (61.77 vs 59.93%), respectively.

TABLE 3(a)
Effect of R. irregularis and inactivated yeast on the concentration
of in Neutral Detergent Fiber Digestibility (NDFd) of red
clover plants one year after the inoculation

| | Trifolium prate | Trifolium prate |
|---|---|---|
| | Cut 3 | Cut 3 |
| | NDFd_30 h | NDFd_48 h |
| | % | % |
| 1 Negative | 58.24 b | 59.93 b |
| control | | |
| 2. R. irregularis | 59.73 a | 61.77 a |
| and inactivated | | |
| yeast | | |
| Tukey's HSD | 0.757 | 0.910 |
| P = .10 | | |
| Standard | 0.455 | 0.547 |
| Deviation | | |
| CV | 0.77 | 0.9 |
| Replicate F | 2.058 | 3.184 |
| Replicate | 0.2842 | 0.1835 |
| Prob(F) | | |
| Treatment F | 21.325 | 22.594 |
| Treatment | 0.0191 | 0.0177 |
| Prob(F) | | |

### Example 2: Effect of Rhizophagus irregularis in Combination with Inactivated Yeast and Bacillus Velezensis on Alfalfa Forage Crop

A study was conducted to assess the effects of seed inoculation by Rhizophagus irregularis in combination with inactivated yeast and Bacillus velezensis on fiber digestibility of alfalfa forage crop.

The forage crop investigated was alfalfa (Medicago sativa, Isabelle variety). Alfalfa seeds were inoculated with the mycorrhizal fungi R. irregularis strain 57891 (BCCM) (LALRISE® PRIME) at a label rate of 100 g/ha (1.5 oz per acre) in combination with inactivated yeast (Lallemand) at the same concentration as in Example 1 and B. velezensis at a label rate of 50 g/ha (0.75 oz per acre). The seeds were mixed with the dry microbial product. Non-mycorrhizal alfalfa seeds and without inactivated yeast and B. velezensis were used as negative control.

Inoculated and non-inoculated alfalfa seeds were sown in 4 blocks of 7.5 square meter (per treatment) with a planting rate of 15 kg/ha. Blocks were arranged in randomized complete field trials.

Trials were harvested two times using commercial equipment at standard timings for forage crop. Both inoculated and non-inoculated forage plants were harvested separately. Representative samples were taken for subsequent analysis. The In Vitro Total, Dry Matter Digestibility (IVTDMD at 30 hours and 48 hours) was quantified at the second cut.

The results are summarized in Table 4. The inoculation of alfalfa seeds with R. irregularis, inactivated yeast and B. velezensis significantly increased the IVTDMD of alfalfa fiber at 30 and 48 hours by 1.5% (80.14 vs 78.99%, p=0.078) and 1.3% (86.13 vs 85.06%, p=0.083), respectively.

TABLE 4
Effect of R. irregularis, inactivated yeast and
B. velezensis on In Vitro Total Dry Matter Digestibility
(IVTDMD) of alfalfa fibers at 30 hours and 48 hours

| | Medicago sativa Alfalfa | Medicago sativa Alfalfa |
|---|---|---|
| | Isabella IVTDMD30 | Isabella IVTDMD48 |
| 1. Negative control | 80.13608779 a | 86.12869171 a |
| 2. R. irregularis + | 79.16361837 b | 85.24495595 b |
| inactive yeast | | |
| 3. R. irregularis + | 78.99849535 b | 85.06423848 b |
| inactive yeast + | | |
| B. velezensis | | |
| LSD P = .10 | 0.841491350 | 0.795873024 |
| Standard Deviation | 0.612423094 | 0.579222852 |
| CV | 0.77 | 0.68 |
| Levene's F | 0.365 | 0.185 |
| Levene's Prob(F) | 0.704 | 0.834 |
| Skewness | −0.6681 | −0.671 |
| Kurtosis | 0.4711 | −0.4912 |
| Replicate F | 8.224 | 10.276 |
| Replicate Prob(F) | 0.0151 | 0.0089 |
| Treatment F | 4.030 | 3.868 |
| Treatment Prob(F) | 0.0777 | 0.0833 |

### Example 3: Effect of Rhizophagus irregularis in Combination with Inactivated Yeast on Corn Crop after a Period of Silage

A study was conducted to assess the effects of seed inoculation by R. irregularis in combination with inactivated yeast on fiber digestibility of corn forage crop after a period of silage.

The forage crop investigated was corn. Corn seeds were inoculated with the mycorrhizal fungi R. irregularis strain 57891 (BCCM) (LALRISE® PRIME) at a label rate of 100 g/ha (1.5 oz per acre) in combination with inactivated yeast (Lallemand) at the same concentration as in Example 1. The seeds were mixed with the dry microbial product. Non-mycorrhizal corn seeds and without inactivated yeast were used as negative control.

Inoculated and non-inoculated corn seeds were sown in 4 blocks of 7.5 square meter (per treatment) with a planting rate of 15 kg/ha. Blocks were arranged in randomized complete field trials.

Trials were harvested two times using commercial equipment at standard timings for corn silage. Both inoculated and non-inoculated forage plants were harvested separately. Subsequently, the harvested plant material was used for producing silage.

Representative samples were taken for subsequent analysis. The following parameters were analyzed according to standard methods: soluble carbohydrates, dry matter, soluble fiber, calcium content, fatty acids total, crude proteins, net energy of lactation (NEL) and milk productivity.

The results are summarized in Table 5. The inoculation of corn seeds with R. irregularis and inactivated yeast followed by a period of silage of the harvested plant material increased the water-soluble carbohydrates (WSC-Sugar) by 48.4% (9.2 vs 6.2%) and the ethanol soluble carbohydrates (ESC-Sugar) by 48.8% (6.4 vs 4.3%) of corn fibers and grains. Further, the concentration of soluble fiber (8.55 vs 7.74% (+10.5%)), calcium content (0.21 vs 0.19% (+10.5%)), fatty acids total (2.49 vs 2.4% (+3.8%)) and crude proteins (8.1 vs 7.6% (+6.6%)) also increased. Adding the mycorrhizal fungi and inactivated yeast increased the amount of net energy of lactation (NEL) by 2.6% (0.78 vs 0.76 mcal/kg) and the milk productivity by 3.6% (3731 vs 3600 lbs/ton).

TABLE 5
Effect of R. irregularis and inactivated yeast on concentrations of soluble
carbohydrates, corn soluble fiber, calcium content, total fatty acids, crude proteins,
net energy of lactation and increase in milk productivity of corn silage

| | Corn | Corn | Corn | Corn | Corn | | | Corn |
|---|---|---|---|---|---|---|---|---|
| | WSC- | ESC- | Soluble | Calcium | Fatty acids | Corn | Corn | Milk |
| | Sugar | Sugar | fiber | content | total | CB | NEL | productivity |
| | % | % | (DM %) | (DM %) | (% DM) | % | Mcal/kg | Lbs/ton |
| 1 Negative | 6.2 | 4.3 | 7.74 | 0.19 | 2.4 | 7.6 | 0.76 | 3600 |
| control | | | | | | | | |
| 2 R. | 9.2 | 6.4 | 8.55 | 0.21 | 2.49 | 8.1 | 0.78 | 3731 |
| irregularis | | | | | | | | |
| and | | | | | | | | |
| inactivated | | | | | | | | |
| yeast | | | | | | | | |

### Example 4: Effect of Rhizophagus irregularis in Combination with Inactivated Yeast on Alfalfa Plants after a Period of Silage

A study was conducted to assess the effects of seed inoculation by R. irregularis in combination with inactivated yeast on fiber digestibility of alfalfa forage crop after a period of silage.

For each randomized experimental plot (four replicates) for the two treatments (control and seeds treated with R. irregularis strain 57891 (BCCM) in combination with inactivated yeast as described in Example 1), at each cutting (four times), a forage harvester was used to gather at least 4 kg of homogenized fresh (green alfafa). The harvested alfalfa was stored in 7 L sealed micro-silos and incubated at controlled 22° C. for 60 days to have the proper conditions for silage fermentation. After the incubation period, a subsample of each silage fermentation micro-silos was sent for NIR analysis and compared with the fresh (green) alfalfa of the same plot.

The results after the second cutting (in July) and silage period are included in Table 6. In summary, it is noted that seeds treated with R. irregularis and inactivated yeast followed by a period of silage of the harvested plants showed an increase in crude protein content (CP) by 4.1% (22.8 vs 21.9%), an increase Relative Feed Value (RFV) by 6.1% (139 vs 131) and a decrease in Acid Detergent Fibers (ADF) by 3.9% (34.7 vs 36.1%).

TABLE 6
Effect of R. irregularis and inactivated yeast on concentrations
of crude protein (CP), Relative Feed Value (RFV) and Acid
Detergent Fibers (ADF) after silage of alfalfa plants

| | Medicago | Medicago | Medicago |
|---|---|---|---|
| | sativa | sativa | sativa |
| | Cut 2 | Cut 2 | Cut 2 |
| | CP | ADF | RFV |
| | %; 0; 100 | %; 0; 100 | %; 0; 100 |
| 1 Negative | 21.9 b | 36.1 a | 131 b |
| control | | | |
| 2. R. irregularis | 22.8 a | 34.7 | 139 a |
| and inactivated | | | |
| yeast | | | |
| Tukey's | 0.26 | 1.33 | 7.9 |
| HSD P = .10 | | | |
| Standard | 0.16 | 0.80 | 4.7 |
| Deviation | | | |
| CV | 0.7 | 2.25 | 3.51 |
| Replicate F | 29.610 | 4.057 | 3.858 |
| Replicate | 0.0099 | 0.1401 | 0.1484 |
| Prob(F) | | | |
| Treatment F | 55.373 | 6.400 | 5.731 |
| Treatment | 0.0050 | 0.0854 | 0.0964 |
| Prob(F) | | | |

After the third cutting in August and the silage period, the analysis of the results shows a significant decrease in Acid Detergent Fibers (ADF) by 5.2% (36.6 vs 38.5%), a significant increase in Neutral Detergent Fiber Digestibility (NDFd_30 h) by 9.1% (50.86 vs 46.63%), a significant increase in Neutral Detergent Fiber Digestibility (NDFd_48 h) by 8.8% (52.35 vs 48.13%) and a significant increase in Water Soluble Carbohydrates (WSC) by 14.1% (4.53 vs 3.97%) (Table 7).

TABLE 7
Effect of R. irregularis and inactivated yeast on concentrations
of crude protein (CP), Relative Feed Value (RFV) and Acid
Detergent Fibers (ADF) after silage of alfalfa plants

| | Medicago | Medicago | Medicago | Medicago |
|---|---|---|---|---|
| | sativa | sativa | sativa | sativa |
| | Cut 3 | Cut 3 | Cut 2 | Cut 3 |
| | ADF | NDFd_30 | NDFd_48 | WSC |
| | %; 0; 100 | %; 0; 100 | %; 0; 100 | %; 0; 100 |
| 1 Negative | 38.5 a | 46.63 b | 48.13 b | 3.97 b |
| control | | | | |
| 2. R. irregularis | 36.5 b | 50.86 a | 52.35 a | 4.53 a |
| and inactivated | | | | |
| yeast | | | | |
| Tukey's HSD | 1.50 | 2.736 | 2.679 | 0.457 |
| P = .10 | | | | |
| Standard | 0.90 | 1.644 | 1.610 | 0.275 |
| Deviation | | | | |
| CV | 2.4 | 3.37 | 3.2 | 6.46 |
| Replicate F | 8.639 | 1.559 | 1.403 | 20.398 |
| Replicate | 0.0549 | 0.3621 | 0.3936 | 0.0169 |
| Prob(F) | | | | |
| Treatment F | 9.836 | 13.206 | 13.695 | 8.172 |
| Treatment | 0.0518 | 0.0359 | 0.0343 | 0.0646 |
| Prob(F) | | | | |

### Further Embodiments of the Invention

The invention further provides:

1. A method for increasing digestibility and/or assimilability and/or energy content of forage plants comprising contacting a plant, a plant part or a plant seed with at least one endophytic fungus species to colonize roots of the plant or a plant grown from the plant seed.

2. A method for increasing silage digestibility and/or energy content comprising contacting a plant, a plant part or a plant seed with at least one endophytic fungus species to colonize the roots of the plant or a plant grown from the plant seed.

3. The method of embodiment 1 or 2, further comprising separately, simultaneously or sequentially contacting the plant, the plant part or the plant seed with a yeast.

4. The method of any one of embodiments 1 to 3, further comprising simultaneously contacting the plant, the plant part or the plant seed with a yeast.

5. The method of any one of embodiments 1 to 4, wherein the yeast is in the form of a live yeast, a dead or inactivated yeast, yeast cell walls, soluble yeast extract or yeast cell wall fractions.

6. The method of any one of embodiments 1 to 5, wherein the yeast is a dead or inactivated yeast.

7. The method of any one of embodiments 1 to 6, wherein the at least one endophytic fungus species is at least one arbuscular mycorrhizal fungus species.

8. The method of embodiment 7, wherein the at least one arbuscular mycorrhizal fungus species is at least one strain of Rhizophagus.

9. The method of embodiment 8, wherein the at least one arbuscular mycorrhizal fungus species is at least one strain of Rhizophagus irregularis.

10. The method of embodiment 9, wherein the at least one strain of Rhizophagus irregularis is contacted with a plant seed.

11. The method of any one of embodiments 1 to 10, wherein the plant, plant part or plant seed is from alfalfa, clover, corn, rice, wheat, barley, oats, rye, millet, buckwheat, quinoa or sesame, preferably wherein the plant seed is from alfalfa, clover or corn.

12. The method of embodiment 11, wherein the at least one strain of R. irregularis is present in an amount of about 0.001% to 50% by weight (w/w) of the combination, formulation or composition and the dead or inactivated yeast is present in an amount of about 0.1% to 99% by weight (w/w) of the combination, formulation or composition.

13. The method of embodiment 11, further comprising separately, simultaneously or sequentially contacting the plant, the plant part or the plant seed with a plant-growth promoting rhizobacteria, preferably wherein said plant-growth promoting rhizobacteria is Bacillus velezensis.

14. Use of at least one strain of Rhizophagus and an inactivated yeast for increasing digestibility and/or assimilability and/or energy content of forage plants.

15. Use of at least one strain of Rhizophagus, an inactivated yeast and Bacillus velezensis for increasing digestibility and/or assimilability and/or energy content of forage plants.

1. A method for increasing digestibility and/or assimilability and/or energy content and/or nutritional value of forage plants comprising contacting a plant, a plant part or a plant seed with at least one endophytic fungus species to colonize roots of the plant or a plant grown from the plant seed, and separately, simultaneously or sequentially contacting the plant, the plant part or the plant seed with a yeast.

2. A method for increasing silage digestibility and/or energy content and/or nutritional value comprising contacting a plant, a plant part or a plant seed with at least one endophytic fungus species to colonize the roots of the plant or a plant grown from the plant seed, and separately, simultaneously or sequentially contacting the plant, the plant part or the plant seed with a yeast.

3. The method of claim 1 or 2, comprising simultaneously contacting the plant, the plant part or the plant seed with at least one endophytic fungus species and a yeast.

4. The method of any one of the preceding claims, wherein the yeast is in the form of a live yeast, a dead or inactivated yeast, yeast cell walls, soluble yeast extract or yeast cell wall fractions.

5. The method of claim 4, wherein the yeast is a dead or inactivated yeast.

6. The method of any one of the preceding claims, wherein the at least one endophytic fungus species is at least one arbuscular mycorrhizal fungus species.

7. The method of claim 6, wherein the at least one arbuscular mycorrhizal fungus species is at least one strain of Rhizophagus.

8. The method of claim 7, wherein the at least one arbuscular mycorrhizal fungus species is at least one strain of Rhizophagus irregularis.

9. The method of claim 8, wherein the strain of Rhizophagus irregularis is 57891 (BCCM).

10. The method of claim 8 or 9, comprising contacting a plant seed with the at least one strain of Rhizophagus irregularis and inactivated yeast.

11. The method of any one of the preceding claims, wherein the plant, plant part or plant seed is from alfalfa, clover, corn, rice, wheat, barley, oats, rye, millet, buckwheat, quinoa or sesame, preferably wherein the plant seed is from alfalfa, clover or corn.

12. The method of any one of the preceding claims, wherein the at least one endophytic fungus species is present in an amount of about 0.001% to 50% by weight (w/w) of a combination, formulation or composition comprising the at least one endophytic fungus species and the dead or inactivated yeast, and the dead or inactivated yeast is present in an amount of about 0.1% to 99% by weight (w/w) of the combination, formulation or composition.

13. The method of claim 12, wherein the at least one endophytic fungus species and inactivated yeast weight ratio is between 0.001 and 100, between 0.01 and 75, between 0.02 and 50, between 0.05 and 25, between 0.05 and 20, between 0.05 and 15 or between 0.05 and 10.

14. The method of any one of the preceding claims, further comprising separately, simultaneously or sequentially contacting the plant, the plant part or the plant seed with a plant-growth promoting rhizobacteria, preferably wherein said plant-growth promoting rhizobacteria is Bacillus velezensis.

15. Use of at least one endophytic fungus species and an inactivated yeast for increasing digestibility and/or assimilability and/or energy content and/or nutritional value of forage plants.

16. Use of at least one endophytic fungus species, an inactivated yeast and Bacillus velezensis for increasing digestibility and/or assimilability and/or energy content and/or nutritional value of forage plants.

17. Use according to claim 15 or 16, wherein the at least one endophytic fungus species is at least one strain of Rhizophagus.

18. Use according to claim 17, wherein the at least one strain of Rhizophagus is Rhizophagus irregularis.

19. Use according to claim 18, wherein the strain of Rhizophagus irregularis is 57891 (BCCM).

20. A forage plant seed inoculated with a combination, formulation or composition comprising at least one endophytic fungus species and yeast.

21. The forage plant seed of claim 20, wherein the yeast is in the form of a live yeast, a dead or inactivated yeast, yeast cell walls, soluble yeast extract or yeast cell wall fractions.

22. The forage plant seed of claim 21, wherein the yeast is a dead or inactivated yeast.

23. The forage plant seed of any one of claims 20-22, wherein the at least one endophytic fungus species is at least one arbuscular mycorrhizal fungus species.

24. The forage plant seed of claim 23, wherein the at least one arbuscular mycorrhizal fungus species is at least one strain of Rhizophagus.

25. The forage plant seed of claim 24, wherein the at least one arbuscular mycorrhizal fungus species is at least one strain of Rhizophagus irregularis.

26. The forage plant seed of claim 25, wherein the strain of Rhizophagus irregularis is 57891 (BCCM).

27. The forage plant seed of claim 25 or 26, inoculated with the at least one strain of Rhizophagus irregularis and inactivated yeast.

28. The forage plant seed of any one of claims 20-27, wherein the forage plant seed is from alfalfa, clover, corn, rice, wheat, barley, oats, rye, millet, buckwheat, quinoa or sesame, preferably from alfalfa, clover or corn.

29. The forage plant seed of any one of claims 20-28, wherein the combination, formulation or composition comprises the at least one endophytic fungus species present in an amount of about 0.001% to 50% by weight (w/w) and the dead or inactivated yeast, and the dead or inactivated yeast is present in an amount of about 0.1% to 99% by weight (w/w).

30. The forage plant seed of claim 29, wherein the at least one endophytic fungus species and inactivated yeast weight ratio is between 0.001 and 100, between 0.01 and 75, between 0.02 and 50, between 0.05 and 25, between 0.05 and 20, between 0.05 and 15 or between 0.05 and 10.

31. The forage plant seed of any one of claims 20-30, further inoculated with a plant-growth promoting rhizobacteria, preferably wherein said plant-growth promoting rhizobacteria is Bacillus velezensis.

32. A method for inoculating a forage plant seed, comprising contacting said forage plant seed with a combination, formulation or composition comprising at least one endophytic fungus species and yeast.

33. The method of claim 32, wherein said combination, formulation or composition, or said forage plant, is as defined in any one of claims 20-31.

34. A method of growing a forage plant, comprising sowing an inoculated forage plant seed according to any one of claims 20-31 and growing a forage plant from said seed.

35. The method of claim 34, further comprising producing silage from said forage plant.

36. A method of producing silage, wherein the silage is produced from a forage plant grown from an inoculated forage plant seed according to any one of claims 20-31.
